(19) United States
(12) Patent Application Publication
(10) Pub. No.: US 2010/0022462 A1
(43) Pub. Date: Jan. 28, 2010

(54) APPETITE SUPPRESSANT AND COLON CLEANSING COMPOSITION

(76) Inventor: Yuan Shi, Little Neck, NY (US)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(21) Appl. No.: 12/180,968

(22) Filed: Jul. 28, 2008

(51) Int. Cl. A61K 31/7048; A61P 3/04

(52) U.S. Cl. 514/25

(57) ABSTRACT

The present invention provides low calorie, high potency, sugar free compositions and methods for making and using the same. In one embodiment, the composition of the present invention can be used to reduce daily food intake and facilitate detoxification and weight loss.

### FIELD OF THE INVENTION

The present invention provides low calorie, high potency, sugar free compositions and methods for making and using the same. In one embodiment, the invention relates to using the composition disclosed herein to reduce daily food intake and facilitate detoxification and weight loss.

### BACKGROUND OF THE INVENTION

Weight loss or gain is a health issue. The normal step to lose body fat is changing your diet. Eat fewer calories, create a mild caloric deficit by monitoring your portion size and making sure you are eating less than you are burning. Eating several small meals throughout the day will boost your metabolism and help you burn more calories. Drink more water, and exercise will burn out calories.

Medical treatment of weight loss includes appetite suppressant medications. These include: DIDREX, TENUATE, SANOREX, MAZANOR, ADIPEX-P, IONAMIN, BONTRIL and MERIDIA. These medications generally come in the forms of tablets or extended-release capsules. Appetite suppressants promote weight loss by tricking the body into believing that it is not hungry or that it is full. They decrease appetite by increasing serotonin or catecholamine, two brain chemicals that affect mood and appetite.

XENICAL (orlistat) is a new class of anti-obesity drugs known as lipase inhibitors. Lipase is the enzyme that breaks down dietary fat. XENICAL interferes with lipase function, decreasing dietary fat absorption by 30 percent. Because the undigested fats are not absorbed, fewer calories are available to the body. This may help in controlling weight gain. The side effects of XENICAL are cramping, diarrhea, flatulence, intestinal discomfort, and leakage of oily stool.

Weight loss medications can help you lose weight, but only if you also cut calories and increase physical activity. Effective weight loss is slow and gradual, and it is a long term task. Moreover, medication does not work for everyone.

Weight loss drugs have side effects, and these drugs are only good for as long as you are taking them. Currently, most weight-loss medications approved by the FDA are for short-term use, meaning a few weeks or months. Hence, there is a need for improved methods for controlling weight loss/gain.

### SUMMARY OF THE INVENTION

The present invention provides a powerful and delicious appetite suppressant that is sugar free and very low in calories. It gives you full feeling and increases bowel movement. This sugar free composition is not like another weight loss supplements that are formulated by a combination of herb.

In one embodiment, the present composition comprises soluble fibers. Soluble fibers help prolong stomach emptying time, it can also lower total cholesterol and LDL cholesterol, therefore reducing the risk of heart disease or increased blood sugar for people with diabetes.

In another embodiment, the present invention also relates to eliminating waste from the body. A clean colon is vital to weight loss and overall health status. Many people today do not fully aware that unclean colon cause many diseases. Diet in fatty food, and consumption of a lot of sugar-based beverage and food all contribute to the process of waste accumulation in the body. If a person's bodily toxic levels increase, stress and other harmful factors can limit the person's everyday life. Often people who need a detoxification treatment experience less energy and have decreased brain function.

Detoxification is a process of getting rid of harmful substances from the body. Detoxifying the body not only help weight loss, it also keep sickness in bay. Currently, there are many colon cleaners on the market, e.g. senna, bentonite clay, cascara sagrada, flax seed, etc.

The present invention provides a food based composition that is sugar free, low calorie, and low glycemic. The composition of the present invention can be used as an appetite suppressant. In another embodiment, the present composition is a colon cleansing composition.

### DETAILED DESCRIPTION OF THE INVENTION

The present invention discloses a food-based composition that is sugar free, low calorie and low glycemic. In one embodiment, the composition comprises a blend of Luo Han Guo whole fruit extract (not the sweet component of mogrosides, such as gorosides IV, mogrosideV and 11-oxo-mogroside), sugar alcohol, soluble fiber and optionally flavor agent. In one embodiment, the composition of the present invention is in the form of syrup. In another embodiment, the composition of the present invention is an appetite suppressant. In another embodiment, the composition can serve as a colon cleansing agent.

Luo Han Guo (Siraitia grosvenori or Momordica grosvenori) is the fruit of the plant Siraitia grosvenorii in the Curcubitaceae family. The fruit is well-known for its sweet taste. There are no reported incidents of negative side effects of Luo Han Guo, which is classified by the American Food and Drug Administration as a GRAS (generally recognized as safe) product. There are no restrictions on consuming the Luo Han Guo fruit or its extracts.

One of ordinary skill in the art would readily prepare extracts from Luo Han Guo fruit using standard procedure and techniques. For example, a method of preparing whole fruit extract from Luo Han Guo fruits comprises (i) cleaning the fruits; (ii) breaking the outer shell or peel of the fruits; (iii) boiling the fruits in water, thereby obtaining an extracted liquid containing solid components of the fruits; and (iv) separating the solid components of the fruits from the extracted liquid, wherein the extracted liquid contains extracts of the peel and pulp of the fruits. In general, the Luo Han Guo fruits are boiled for about 1 to 2 hours. The extracted liquid containing solid components of the fruits can be further acidified to about pH 4.0-5.0 with an edible acid before digesting with pectinase to remove pectin. The pectinase enzyme is then denatured by heating after the digestion. Preferably, the final extracted liquid without the solid components of the fruits is further concentrated before formulating.

Dietary fibers are usually divided according to whether they are water-soluble or not. Both types of fiber are present in all plant foods, with varying degrees of each according to a plant's characteristics. Insoluble fiber possesses passive water-attracting properties that help to increase bulk, soften stool and shorten transit time through the intestinal tract. Soluble fiber undergoes metabolic processing via fermentation, yielding end-products with broad and significant health effects. Dietary fiber consists of non-starch polysaccharides such as cellulose and many other plant components such as dextrins, inulin, lignin, waxes, chitins, pectins, beta-glucans, fructooligosaccharides and oligosaccharides.

Sugar alcohols are neither sugars nor alcohols. They are carbohydrates with a chemical structure that partially resembles sugar and partially resembles alcohol, but they don't contain ethanol as alcoholic beverages do. They are incompletely absorbed and metabolized by the body, and consequently contribute fewer calories. The commonly used sugar alcohols include sorbitol, mannitol, xylitol, maltitol, maltitol syrup, lactitol, erythritol, isomalt and hydrogenated starch hydrolysates. Their calorie content ranges from 1.5 to 3 calories per gram compared to 4 calories per gram for sucrose or other sugars. Most are approximately half as sweet as sucrose; maltitol and xylitol are about as sweet as sucrose.

Many weight loss products claim to be natural or herbal, but this does not necessarily mean that they are safe. The ingredients of those products may interact with drugs or may not be clearly understood. The present invention provides a novel food-based composition for appetite suppressant and colon cleansing.

To achieve weight loss requires lifestyle changes, including diet and nutrition, physical activity. Before one want to lose weight or maintain a healthy weight, it's important to understand the connection between the calories one's body takes in and the calories one's body uses. The composition of the present invention suppresses food intake and gives a full feeling to cut calorie intake. At the same time the composition of the present invention can also increase bowel movement.

In one embodiment, the sugar free composition of the present invention comprises the following components: whole Luo Han Guo extract; maltitol syrup; inulin; and water.

In one embodiment, the sugar free composition of the present invention is produced by a method comprising (a) mixing 15% inulin with 20% water; (b) adding 1.0% Whole Luo Han Guo extract into the solution from (a); (c) adding 64% maltitol syrup into solution from (a); (d) heating the mixture to 80-100° C. with agitation; and (e) maintaining the temperature and agitate for 30-60 min.

The present invention provides a sugar free composition comprising sugar alcohol, soluble fiber, and Luo Han Guo concentrate. In general, examples of sugar alcohols include, but are not limited to, maltitol syrup, maltitol, sorbitol, mannitol, xylitol, lactitol, ismalt, and hydrogenated starch hydrolysates, whereas the soluble fiber can be extracted from fruits or vegetables such as bananas, onions, chicory root, garlic, asparagus, barley, wheat, jícama, tomatoes, leeks, konjac, oat, oat bran, dried beans, peas, and flax seed.

In one embodiment, the composition comprises from about 35% to about 70% by weight of sugar alcohol. In another embodiment, the composition comprises from about 5% to about 30% by weight of soluble fiber. In yet another embodiment, the composition comprises from about 3% to about 10% by weight of the Luo Han Guo concentrate.

In one embodiment, the composition is formulated as a syrup.

The present invention also provides a method of making a sugar free composition useful as an appetite suppressant, comprising the steps of: (a) mixing water with soluble fiber; (b) adding Luo Han Guo concentrate into sugar alcohol with water; (c) adding the solution obtained from (b) into the solution obtained from (a); and (d) heating the mixture to 80-100° C. with agitation, thereby obtaining a sugar free composition useful as an appetite suppressant.

The present invention also provides a method of obtaining weight loss in a subject, comprising the step of administering the composition described herein to said subject, wherein the composition acts as an appetite suppressant. In one embodiment, the composition further acts as a natural colon cleansing agent in the subject.

What is claimed is:

1. A sugar free composition comprising sugar alcohol, soluble fiber, and Luo Han Guo concentrate.

2. The composition of claim 1, wherein the sugar alcohol is selected from the group consisting of maltitol syrup, maltitol, sorbitol, mannitol, xylitol, lactitol, ismalt, and hydrogenated starch hydrolysates.

3. The composition of claim 1, wherein the soluble fiber is food source fiber.

4. The composition of claim 3, wherein the fiber is extracted from fruits or vegetables selected from the group consisting of bananas, onions, chicory root, garlic, asparagus, barley, wheat, jícama, tomatoes, leeks, konjac, oat, oat bran, dried beans, peas, and flax seed.

5. The composition of claim 1, wherein the composition comprises from about 35% to about 70% by weight of sugar alcohol.

6. The composition of claim 1, wherein the composition comprises from about 5% to about 30% by weight of soluble fiber.

7. The composition of claim 1, wherein the composition comprises from about 3% to about 10% by weight of the Luo Han Guo concentrate.

8. The composition of claim 1, wherein the composition is formulated as a syrup.

9. A method of making a sugar free composition useful as an appetite suppressant, comprising the steps of:
a. mixing water with soluble fiber;
b. adding Luo Han Guo concentrate into sugar alcohol with water;
c. adding the solution obtained from (b) into the solution obtained from (a); and
d. heating the mixture to 80-100° C. with agitation, thereby obtaining a sugar free composition useful as an appetite suppressant.

10. The method of claim 9, wherein the sugar alcohol is selected from the group consisting of maltitol syrup, maltitol, sorbitol, mannitol, xylitol, lactitol, ismalt, and hydrogenated starch hydrolysates.

11. The method of claim 9, wherein the fiber is extracted from fruits or vegetables selected from the group consisting of bananas, onions, chicory root, garlic, asparagus, barley, wheat, jícama, tomatoes, leeks, konjac, oat, oat bran, dried beans, peas, and flax seed.

12. The method of claim 9, wherein the composition comprises from about 35% to about 70% by weight of sugar alcohol.

13. The method of claim 9, wherein the composition comprises from about 5% to about 30% by weight of soluble fiber.

14. The method of claim 9, wherein the composition comprises from about 3% to about 10% by weight of the Luo Han Guo concentrate.

15. The method of claim 9, wherein the composition is formulated as a syrup.

16. A method of obtaining weight loss in a subject, comprising the step of administering the composition of claim 1 to said subject, wherein the composition acts as an appetite suppressant.

17. The method of claim 16, wherein the composition further acts as a natural colon cleansing agent.
